(19) United States
(12) Patent Application Publication
(10) Pub. No.: US 2019/0206547 A1
(43) Pub. Date: Jul. 4, 2019

(54) PORTABLE ELECTRONIC DEVICE FOR DATA COLLECTION, AND RELATED CHARGING STATION AND SYSTEM

(71) Applicant: Auxilia Limited, Sheung Wan (HK)

(72) Inventors: Eimund Loo, Midlevels (HK); Sedonia Suet Man Shu, Midlevels (HK)

(21) Appl. No.: 15/758,385

(22) PCT Filed: Jan. 3, 2018
(86) PCT No.: PCT/CN2018/070075

§ 371 Date: Mar. 8, 2018

(30) Foreign Application Priority Data
Jan. 3, 2018 (CN) PCT/CN2018/070075
Jan. 6, 2018 (HK) 17100178.5

(51) Int. Cl. G16H 40/20; H02J 7/00; H03K 17/967; G08B 5/22; G08B 6/00; G08B 3/10; H04W 4/80

(52) U.S. Cl. CPC G16H 40/20; H02J 7/0052; H03K 17/967; H04W 4/80; G08B 6/00; G08B 3/10; G08B 5/22

(57) ABSTRACT

A portable electronic device includes a housing with a display displaying instructions or data, and a plurality of actuators and touch panel receiving user input. The user input is associated with one or more of: time-related or task-related events associated with care needs of a subject, biological events related to the subject, and biological measurements related to the subject. The housing contains a processor operably connected with the plurality of actuators and the touch panel; a memory operably connected with the processor; and a wireless communication module operably connected with the memory and/or the processor. During operation, the processor is operable to process the user input received at the plurality of actuators and the touch panel for recording the respective event or measurement. The memory is arranged to store the recorded event or measurement as data for transmission through the wireless communication module to an external electronic device

### TECHNICAL FIELD

The present invention relates to a portable electronic device and particularly, although not exclusively, to a portable, handheld electronic device that facilitates data collection in settings associated with a subject (e.g., infant) that requires care.

### BACKGROUND

Caregivers are usually required to record time and duration of events related to the subject they care for. For example, a caregiver who provides care for an infant may be required to record bottle feeding times, bottle feeding volume, breast feeding times, breast feeding volume, colour or times of bowel movements of the infant. These data may be useful for monitoring and assessing infant growth and development.

Conventionally, the above recording is performed by the caregiver using a paper diary (e.g., writing notes in a logbook), an electronic diary (e.g., entering data through a general or dedicated mobile phone application), or both. Sometimes, the caregiver, being careless or because of other distractions, may have forgotten to perform a care-related task or to record a care-related task that has been performed, and so some of the data may not have been recorded. As a result, the data record is incomplete and sometimes inaccurate. Such data record is at best good enough for detecting instant irregularities, but is not suitable for analysis or observations of mid- to long-term trends.

With the rapid advancement in health care technologies and big data analytics, a more complete and accurate record of data would allow more information to be harnessed and utilized, thereby better tailoring care for individual subjects.

### SUMMARY OF THE INVENTION

In accordance with a first aspect of the present invention, there is provided a portable electronic device comprising: a housing with a display for displaying instructions or data; and a plurality of actuators and a touch panel, for receiving user input associated with one or more of: time-related or task-related events associated with care needs of a subject, biological events related to the subject, and biological measurements related to the subject; the housing contains a processor operably connected with the plurality of actuators and the touch panel; a memory operably connected with the processor; and a wireless communication module operably connected with the memory and/or the processor; wherein during operation the processor is operable to process the user input received at the plurality of actuators and the touch panel for recording the respective event or measurement; and wherein the memory is arranged to store the recorded event or measurement as data for transmission through the wireless communication module to an external electronic device.

In one embodiment of the first aspect, the task-related event, time-related event, biological event, and biological measurement comprises any of: a diaper change event; a feeding event; a sleeping event; and a medication event.

In one embodiment of the first aspect, the diaper change event comprises any of: a time at which a diaper change occurs, and a stool or urination event.

In one embodiment of the first aspect, the stool or urination event comprises any of: stool colour, stool quantity, stool quality, urine colour, urine quantity, and urine quality.

In one embodiment of the first aspect, the feeding event comprises any of: a time at which feeding begins, a time at which feeding ends, a feeding volume, and a feeding source.

In one embodiment of the first aspect, the sleeping event comprises: a time at which sleeping begins or a time at which sleeping ends.

In one embodiment of the first aspect, the medication event comprises any of: a time at which medication is taken, a medication volume, and a medication source.

In one embodiment of the first aspect, the user input is further associated with a breastmilk expression event comprising any of: a time at which the expression occurs, and a volume of the breastmilk expressed.

In one embodiment of the first aspect, the user may manipulate the touch panel to provide an input by tapping, pinching, sliding, and/or swiping his or her fingers on the touch panel.

In one embodiment of the first aspect, the plurality of actuators are operable as selection keys during operation. In one example, during operation the display may display different settings or parameters, and the user have to actuator the actuators to select a setting or parameter.

In one embodiment of the first aspect, the housing further comprising an alarm module operably coupled with the processor for providing an alarm. The alarm module may comprise one or more of: a visual alarm module, a tactile alarm module, and an audible alarm module.

In one embodiment of the first aspect, the visual alarm module comprises the display or one or more LEDs. The one or more LEDs may form an LED bar. The one or more LEDs may be of different colour for indicating different information.

In one embodiment of the first aspect, the tactile alarm module comprises a vibration motor. The tactile alarm module may comprise an eccentric rotating mass, a linear resonant actuator or a piezo.

In one embodiment of the first aspect, the audible alarm module comprises a speaker or a buzzer.

In one embodiment of the first aspect, during operation, the alarm module is triggered to provide an alarm based on the time-related reminder data. The time-related reminder data may be a predetermined time set by the user at which the alarm will be actuated. The alarm may be a visual alarm, e.g., lighting up of one or more LEDs of a particular colour or colour sequence, flashing of one or more LEDs, or lighting up of the screen. The alarm may be an audible alarm, which may be a played sound, message, tone, beep, etc., and can be continuous or intermittent. The alarm may be a tactile alarm, including a continuous or intermittent vibration that can be felt by the user.

In a preferred embodiment of the first aspect, the wireless communication module comprises a Bluetooth Low Energy communication module arranged to communicate with the external electronic device using Bluetooth Low Energy communication protocol. In other embodiments, other communication modules/protocols, for example ZigBee, NFC, RFID and Wi-Fi, may also be used. Preferably, the communication is secured and encrypted.

In one embodiment of the first aspect, the plurality of actuators are momentary actuators. In other words, the user need not continuously hold onto or actuate the actuator in order to provide an input.

In one embodiment of the first aspect, the memory comprises non-volatile memory. The non-volatile memory may store the time data, regardless of the power state of the device. In some examples, the memory may comprise or further comprise volatile memory.

In one embodiment of the first aspect, the display is a LCD screen. In some example, the display screen can be a touch-sensitive screen.

In one embodiment of the first aspect, the housing further contains a power source. Preferably, the power source comprises a battery. The battery is preferably rechargeable.

In another embodiment of the first aspect, the housing contains a charging circuit operably connected with the battery. Conductive charging pins operably connected with the charging circuit, adapted for receiving power from an external power source, may be arranged on the housing.

In one embodiment of the first aspect, the housing has a substantially oblong profile, and the plurality of actuators are arranged along a perimeter of the housing. In one example, the display is arranged on a front side of the housing, and the plurality of actuators are arranged along a peripheral edge of the housing.

In one embodiment of the first aspect, the subject is an infant. In another embodiment of the first aspect, the subject may be an elderly, a patient, or any other subject that require care. The subject is preferably human but may also be other animals.

In accordance with a second aspect of the present invention, there is provided a charging station for the portable electronic device of any one of the preceding claims, comprising: a body arranged to provide a mount for the portable electronic device; the body containing: a processor for processing data; and a wireless communication module arranged to communicate data with the wireless communication module of the portable electronic device and to communicate data with a server.

In one embodiment of the second aspect, the body further contains a charging circuit, arranged to receive power, for charging a power source, e.g., battery of the portable electronic device. Preferably, the charging circuit includes charging pins or contacts that correspond to charging contacts or pins formed on the portable electronic device.

In one embodiment of the second aspect, the body further contains a memory for storing data received from the portable electronic device and/or data received from the server.

In one embodiment of the second aspect, the wireless communication module comprises a Bluetooth Low Energy communication module arranged to communicate data with the portable electronic device using Bluetooth Low Energy communication protocol. In other embodiment, other communication modules/protocols, for example, ZigBee, NFC, RFID and Wi-Fi, may also be used. Preferably, the communication is secured and encrypted.

In one embodiment of the second aspect, the wireless communication module comprises a Wi-Fi communication module arranged to communicate data with the server using Wi-Fi communication protocol. In other embodiment, other communication modules/protocols, for example ZigBee, Bluetooth, or other telecommunication modules/protocols (e.g., 2G, 3G, 4G, LTE) may also be used. Preferably, the communication is secured and encrypted.

In accordance with a third aspect of the present invention, there is provided a system comprising: a portable electronic device in accordance with the first aspect of the present invention; and an external electronic device operably connected with the portable electronic device.

In one embodiment of the third aspect, the external electronic device is the charging station in accordance with the second aspect of the present invention or is a mobile device. The mobile device may be a computer, a mobile phone, a tablet, etc., that may include different hardware and software components. The mobile device may be operable to store, process, or analyze data received from the portable electronic device. The mobile device may be operable to transmit commands, program, or data to the portable electronic device.

In one embodiment of the third aspect, the system further comprises a server operably connected with the charging station, wherein the server is operable to process or analyze data received from the charging station. The server may include a database for storing data. The server may be arranged in a cloud computing network.

It is an object of the present invention to address the above needs, to overcome or substantially ameliorate the above disadvantages or, more generally, to provide an improved portable, handheld, or wearable electronic device that facilitates data collection in settings associated with a subject that requires care.

### BRIEF DESCRIPTION OF THE DRAWINGS

Embodiments of the present invention will now be described, by way of example, with reference to the accompanying drawings in which:

FIG. 1A is the front view of a portable electronic device in accordance with one embodiment of the present invention;

FIG. 1B is the side perspective view of the portable electronic device of FIG. 1A, and illustrating a connection of a strap of the device;

FIG. 2 is a functional block diagram of a portable electronic device in accordance with one embodiment of the present invention;

FIG. 3 is a perspective view of a charging station of the portable electronic device in accordance with one embodiment of the present invention, with the portable electronic device mounted to the charging station;

FIG. 4 is a functional block diagram of a charging station in accordance with one embodiment of the present invention;

FIG. 5 illustrates a system comprising the portable electronic device of FIGS. 1A-1B and a charging station of FIG. 3;

FIG. 6A is a front view of a portable electronic device in accordance with another embodiment of the present invention;

FIG. 6B is a perspective view of the housing of the portable electronic device of FIG. 6A;

FIG. 6C is another perspective view of the housing of the portable electronic device of FIG. 6A;

FIG. 7A is a perspective view of a charging station of the portable electronic device in accordance with another embodiment of the present invention;

FIG. 7B is a perspective view of the charging station of FIG. 7A, with the portable electronic device mounted to the charging station; and

FIG. 8 illustrates a system comprising the portable electronic device of FIGS. 6A-6C and the charging station of FIGS. 7A-7B.

### DETAILED DESCRIPTION OF THE PREFERRED EMBODIMENT

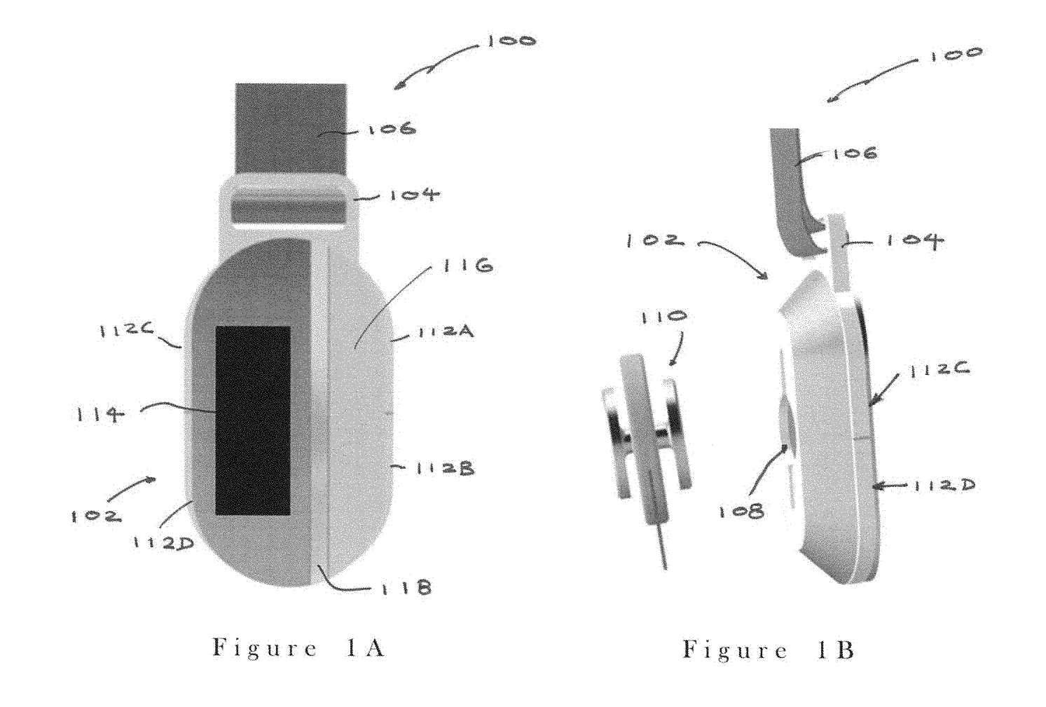

FIGS. 1A and 1B show an exemplary portable electronic device 100 in accordance with one embodiment of the present invention. The device 100 in this embodiment includes a housing 102 having a substantially oblong profile, and a strap mount 104 arranged at one side of the housing. The strap mount 104 includes a buckle with two openings suitable for receiving a strap 106. The strap mount 104 is preferably integrally formed with the housing 102. As shown in FIGS. 1A and 1B, a strap 106 is mounted to the strap mount 104. A blind-recess 108, preferably sized to receive an anchor disc of a strap 106, is formed at the back side of the housing 102. In one example, the portable electronic device 100 includes a strap 106 with an anchor disc no at one end, and the strap 106 is connected at one end to the strap mount 104 and at another end with the recess 108 through the anchor disc 110. As a result, the strap 106 and the housing 102 together define a close loop, allowing the device 100 to be worn by a user. In another example, the strap 106 may be only connected to the strap mount 104, not to any other parts of the housing 102. In one embodiment, the blind-recess 108 may also be used to receive a wall-mounted hook or other attachment means for mounting or hanging the device 100.

As shown in FIG. 1A, four buttons 112A-112D of like form are arranged along a peripheral edge of the housing 102. The two pairs of buttons 112A, B, 112C, D are arranged substantially symmetrically about a long axis of the housing 102. Preferably, the buttons 112A-112D are momentary actuators. The buttons 112A-112D are each arranged to be pressed by the user to receive a user input. The buttons 112A-112D are arranged to be associated with a type of task-related event related to care needs of a subject. For an infant subject, the task-related event related to care needs may be a diaper change event, a feeding event, a sleeping event, or a medication event. The diaper change event may include a time at which a diaper change occurs. The feeding event may include a time at which feeding begins or a time at which feeding ends. The sleeping event may include a time at which sleeping begins or a time at which sleeping ends. The medication event may include the time at which medication is taken.

The associations between the buttons 112A-112D and the types of task-related event related to care needs of a subject may be programmed by the user. These associations may also be re-programmable. The buttons 112A-112D may further be operable as selection keys during operation. For example, in operation the display 114 of the device 100 may display different settings or parameters, and the buttons can be used to select a setting or parameter displayed.

Again referring to FIGS. 1A and 1B, the front side of the housing 102 is arranged with a display 114, a touch panel 116, and a light indicator bar 118 separating the display 114 and the touch panel 116. The display 114 is preferably a LCD display screen, and it may be touch-sensitive for receiving user input. In operation, the display 114 may display data, selections, instructions, etc., in the form of text or graphics. The touch panel 116 is operable, during operation, to receive user input associated with other time-related or task-related events associated with care needs of a subject, biological events related to the subject, or biological measurements related to the subject. These events and measurements may include feeding volume, feeding source (e.g., formula, breastmilk, breastfeed-left, breastfeed-right, solids), medication volume, medication source, stool/urine colour, and stool/urine quality or quantity. The touch panel 116 may receive different inputs from the user, in the form of tapping, pinching, sliding, or swiping, etc. The light indicator bar 118 in the present embodiment is an LED array that includes LEDs of different colours, e.g., red, green, and blue. The light indicator bar 118 is arranged to provide a visual alarm or to display different status of the device (e.g., low power state, error state, etc.).

The portable electronic device 100 of FIGS. 1A and 1B further includes a number of internal components (not visible from FIGS. 1A and 1B). In the present embodiment, the device includes a microprocessor, a memory, and a wireless communication module that are operably connected with each other through a main bus.

During operation, the microprocessor is arranged to determine time-points related to respective task-related events based on manipulation of the respective buttons 112A-112D so as to collect time data. For example, when a diaper change event occurs, the user can press the button arranged to associate with diaper change event, and the microprocessor will determine the time (including date and time) at which the button 112A-112D is pressed to record the event. Preferably, the user only needs to press the button 112A-112D once, momentarily, to record the event. The microprocessor is further arranged to process program instructions for display on the display 114 to interact with the user, and to process user inputs received through the buttons 112A-112D or the touch panel 116.

The memory in the present embodiment comprises a non-volatile memory for storing data (e.g., time data) inputted by the user. Data stored in the non-volatile memory is substantially unaffected by the power state of the device. In one example, when the user presses a button 112A-112D and the microprocessor determines the time at which the button 112A-112D is pressed, the time data (e.g., in the form of a time stamp, associated with respective event identifier) is stored in the memory. In another example, when the user manipulates the touch panel 116 to record an event or measurement entry (other time-related or task-related events associated with care needs of a subject, biological events related to the subject, or biological measurements related to the subject), the corresponding data or entry is stored in the memory. The memory in the present embodiment is arranged to store historical data, as long as the memory capacity has not been exceeded. In one example, the memory capacity is sufficient for storing event records of a few days or one week. Data stored in the memory may be transferred to an external electronic device and hence to a server through the wireless communication module of the device 100. The memory in the present embodiment may also comprise other volatile or non-volatile memory for storing program instructions or parameters.

The wireless communication module in the present embodiment is a Bluetooth Low Energy (BLE) communication module arranged to communicate with the external electronic device using Bluetooth Low Energy communication protocol. BLE is preferred in the present embodiment because of its robustness, simplicity, and low power operation. The BLE communication module is arranged to communicate data in real time to and from the device, but in some cases, it may communicate data at regular time intervals. In one example, the BLE communication module is arranged to communicate in real time the data or the new data stored in the memory to an external electronic device and hence to a server. The BLE communication module may also receive commands, instructions, or data from an external electronic device to affect the function of the device. Preferably, the communication is secured and encrypted.

The portable electronic device 100 of FIGS. 1A and 1B further includes, internally, a power source. The power source may be a disposable battery that is replaceable or may be a rechargeable battery. In the present embodiment, the battery is rechargeable and has Lithium-based chemistry. The power source includes a wireless charging circuit with a charging coil in electric connection with the battery. The charging coil may receive energy, inductively, from an external charging station. The power source in the present embodiment is operably connected with different functional modules of the device so as to power the different functional modules.

In the present embodiment, an alarm module is further provided inside the portable electronic device 100. The alarm module is arranged to be controlled by the processor to provide alarm function—to remind the user of the need to perform a specific task at a specific moment. The alarm module in the present embodiment includes a visual alarm module, a tactile alarm module, and an audible alarm module. The visual alarm module includes the display and the light indicator bar, arranged to provide visual stimulus (e.g., flashing of lights, turning on/off particular color of light, etc.) as an alarm. The tactile alarm module includes a vibration motor arranged in the device to provide alarms in the form of continuous or intermittent vibrations. The audible alarm module includes a speaker arranged to produce a sound (e.g., tone, beep, speech, music, etc.), continuous or intermittent, that serves as an alarm. The three alarm modules may operate individually or together at the same time, to provide different alarms. In one example, during operation, the BLE communication module is operable to receive time-related reminder data, e.g., in the form of a time stamp and an associated event identifier, from an external electronic device. The data received is processed by the microprocessor, optionally stored in the memory, and the alarm module(s) are triggered to provide an alarm at a specified time. In an alternative embodiment, the alarm may only be set by the user locally at the device 100, i.e., the device 100 is not arranged to receive time-related reminder data from other external electronic devices through the communication module.

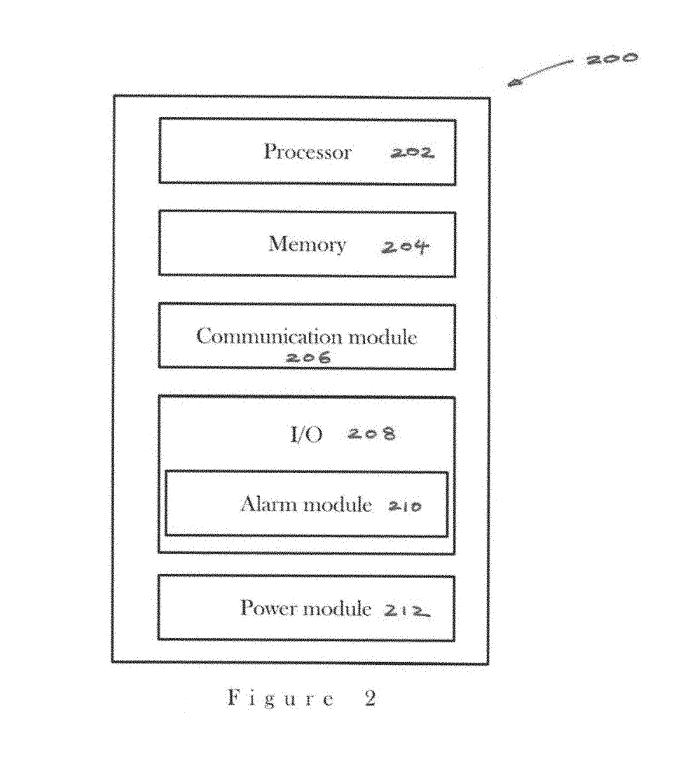

FIG. 2 shows the functional block diagram of an exemplary portable electronic device 200 in accordance with one embodiment of the present invention. In this embodiment the device 200 includes a processor 202, a memory 204, a communication module 206, input/output (I/O) means 208 with an alarm module 210, and a power module 212, connected with one another, directly or indirectly, through one or more data bus and power bus (not shown). In one example, the data bus may be a Peripheral Component Interconnect (PCI) such as PCI Express, a Universal Serial Bus (USB), and/or an optical bus structure.

The processor 202 may be a CPU, an MCU, or a microprocessor for processing input, output, program data, or instructions. The memory 204 may include a non-volatile unit (such as ROM, EPROM, EEPROM and flash memory), a volatile memory unit (such as RAM), or both, arranged to store data, parameters, program codes, or instructions. A suitable operating system may further be installed in the memory 204.

The communication module 206 is a wireless communication module for establishing one or more wireless communication links with other external computing devices such as a handheld computing devices or mobile phone. Preferably, the wireless communication module 206 is a Bluetooth Low Energy (BLE) communication module arranged to communicate with the external electronic device using BLE communication protocol. In other embodiments, other communication modules/protocols, for example ZigBee, NFC, RFID and Wi-Fi, may also be used. In some embodiments, the communication is secured and encrypted. The communication module 206 is operable to communicate commands, instructions, information and/or data with one or more external electronic devices.

The input means 208 of the device 200 comprises a plurality of actuators, which may be in the form of buttons, switches, etc., arranged to be actuated by the user to receive a user input. The actuators may be momentary actuators such that the user need not continuously hold onto the actuator in order to provide an input. In one embodiment each of the actuators may be arranged to be associated with a type of task-related event related to care needs of a subject. In one example, the associations between the actuators and the types of task-related event related to care needs of a subject are preset by the device manufacturer. In another example, the associations are programmable by the user, for example, by transmitting new program codes or instructions to the device 200 through the communication module 206 to overwriting existing program codes or instructions. Preferably, the actuators are also arranged to serve other functions, e.g., to be used as selection keys. The input means 208 of the device 200 may further comprise one or more displays such as a LCD display, a LED display, or any other suitable display that may or may not be touch sensitive. The input means 208 of the device 200 may further comprise one or more touch panels, arranged to receive user input, which may be in the form of tapping, pinching, sliding, or swiping on the touch panel. In one embodiment, the touch panel may have display function.

The I/O means 208 may further include an alarm module 210. In one embodiment, the alarm module 210 may be a visual alarm module having a display or one or more LEDs. The one or more LEDs may be of different colour and they may form an LED bar, arranged to provide continuous or intermittent visual stimulus. In one embodiment, the alarm module 210 may be a tactile alarm module that includes a vibration motor, an eccentric rotating mass, a linear resonant actuator, or a piezo, arranged to provide continuous or intermittent vibrations. In one embodiment, the alarm module 210 may be an audible alarm module that includes a speaker or a buzzer, arranged to provide continuous or intermittent sound. In some embodiment, the alarm module 210 may be a combination of any two or all of the above three alarm modules.

The power module 212 in the device 200 preferably includes one or more batteries that are rechargeable. The rechargeable battery may be charged wirelessly, e.g., through induction, or be charged using power cables (in which case a charging port or charging pins may be provided on the housing). In another embodiment, the power module 212 includes one or more batteries that may be replaceable, received in a battery receptacle of the device 200. In some embodiments of the present invention, the device 200 may be operable in different power states, e.g., a normal operation state, a reduced power state, and an off state. For example, during normal operation, the device 200 may be in normal operation state. However, if the device 200 has been idle for 15 minutes, it may enter a reduced operating state thereby to reduce power consumption. If the device 200 has been idle for a prolonged period, say 1 day, then it may be turned off automatically. Preferably, the power state of the device 200 is shown through the input/output means 208, e.g., on the display or through an LED bar, so that the user can readily determine whether the battery needs to be recharged or replaced.

In the present embodiment, during operation, the processor 202 is operable to determine time-points related to respective task-related events based on actuations of the respective actuator, to collect time data. For example, when an actuator is actuated, the processor 202 determines the time at which the actuator is actuated and the task-related events associated with that actuator. The result will be recorded in the memory 204, and will then be transmitted to an external electronic device through the wireless communication module 206, in real time or at regular time intervals. In one embodiment, the task-related event related to care needs of a subject comprises any of: diaper change event, feeding event, sleeping event, and medication event. The diaper change event may include a time at which a diaper change occurs. The feeding event may include a time at which feeding begins or a time at which feeding ends. The sleeping event may include a time at which sleeping begins or a time at which sleeping ends. The medication event may include the time at which medication is taken. In some embodiments, the user may use the actuators to input or record other information, e.g., feeding volume, feeding source, medication volume, medication source, stool/urine colour, and stool/urine quality or quantity. The device 200 may display suitable instructions that allow the user to enter this information through the actuators. For example, to enter feeding volume, the display may provide, through text or images displayed on the display, the user with such a selection to enter feeding volume, and the user may use two actuators for setting the volume (one actuator for increase, one actuator for decrease), and use a third actuator to confirm the setting.

In the embodiment that the device 200 includes a touch panel, the touch panel, alone or in combination with the actuators, is operable to receive user input associated with one or more of events related to care needs of a subject, biological event related to the subject, and biological measurement related to the subject. These may include height, weight, feeding volume, feeding source, medication volume, medication source, stool/urine colour, and stool/urine quality or quantity. In one example, to enter feeding volume, the display may provide, through text or images displayed on the display, the user with such a selection to enter feeding volume, and the user may slide his or her finger on the touch panel to make a selection then tab on the panel to confirm the data entry. In another example, the user may slide his or her finger on the touch panel to make a selection then press on of the actuators to confirm the data entry.

In some embodiments, during operation, the wireless communication module 206 is operable to receive time-related reminder data from an external electronic device, and the alarm module 210 is triggered to provide an alarm based on the time-related reminder data. For example, the user may use an application on a mobile phone (external electronic device) to set an alarm to remind himself or herself to perform a specific task at a particular moment. The alarm set by the user may be transmitted to the device 200 of the present invention, and may be stored in the memory 204. The processor 202 then actuates the alarm module 210 at the time set by the alarm.

In some embodiments, the portable electronic device 200 may include further functional modules operably connected with the other modules in the device 200. For example, the device 200 may include a GPS module for tracking geographical location of the user wearing the device 200.

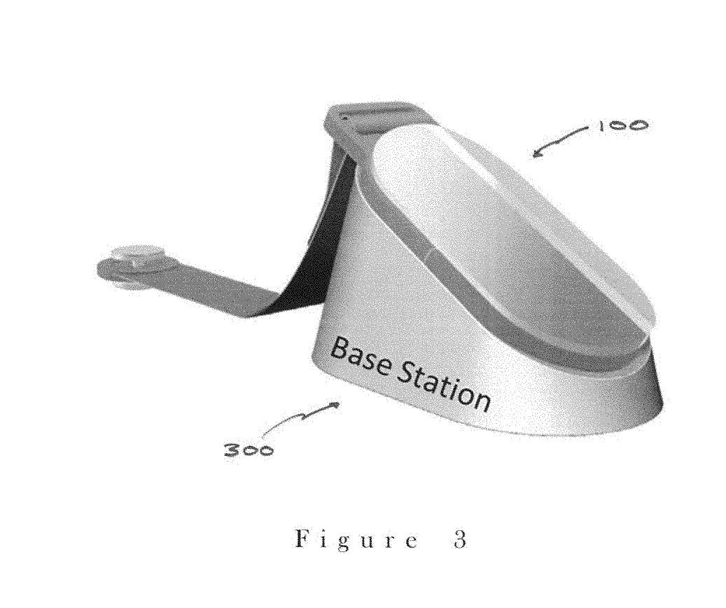

FIG. 3 shows an exemplary base station 300 suitable for charging the portable electronic device 100, 200 of the present invention. In FIG. 3, the portable electronic device 100, 200 is shown resting on a top surface of the base station 300.

The base station 300 in the present embodiment includes a body arranged to provide a mount for the portable electronic device 100, 200. The mount, not shown, may be a mechanical or magnetic mount. In one example, a port may be formed on the base station 300 to engage with the housing of the electronic device 100, 200 through male and female mating features, snap fit, bayonet coupling, etc. In another example, the base station 300 may include a magnet for coupling with a corresponding magnet, metallic or magnetic part of the housing of the electronic device 100, 200. The base station 300 includes, internally, a processor for processing data and one or more wireless communication modules arranged to communicate data. The wireless communication module(s) may communicate with the wireless communication module 206 of the portable electronic device 100, 200 and may further communicate data with a remote server.

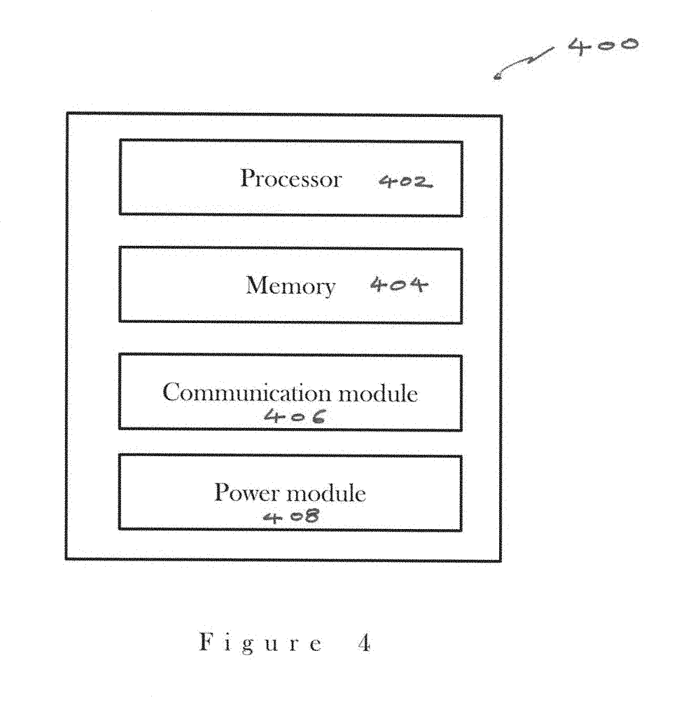

FIG. 4 shows a functional block diagram of an exemplary base station 400 in accordance with one embodiment of the present invention. In this embodiment the base station 400 includes a processor 402, a memory 404, a communication module 406, and a power module 408, connected with one another, directly or indirectly, through one or more data bus and power bus (not shown). In one example, the data bus may be a Peripheral Component Interconnect (PCI) such as PCI Express, a Universal Serial Bus (USB), and/or an optical bus structure.

In the present invention, the processor 402 may be a CPU, an MCU, or a microprocessor. The memory 404 may include a volatile memory unit (such as RAM), a non-volatile unit (such as ROM, EPROM, EEPROM and flash memory) or both, arranged to store data received from the portable electronic device 100, 200 and/or data received from a server.

The communication module 406 may include one or more wireless communication modules arranged to communicate data with the portable electronic device 100, 200 and an external server. Preferably, the wireless communication module 406 comprises a Bluetooth Low Energy (BLE) communication module arranged to communicate data with the portable electronic device 100, 200 using Bluetooth Low Energy (BLE) communication protocol. In some embodiments, other communication modules/protocols, for example, ZigBee, NFC, RFID and Wi-Fi, may also be used for communicating data with the portable electronic device 100, 200. Preferably, the communication between the station 400 and the portable electronic device 100, 200 is secured and encrypted. In a preferred embodiment of the present invention, the wireless communication module 406 further comprises a Wi-Fi communication module arranged to communicate data with the server using Wi-Fi communication protocol. In some embodiments, other communication modules/protocols, for example ZigBee, Bluetooth, or other telecommunication modules/protocols (e.g., 2G, 3G, 4G, LTE) may also be used for communicating data with the server. Preferably, the communication between the station 400 and the server is secured and encrypted.

In one embodiment, the station 400 includes a power module 408 that includes a charging circuit with a charging coil, arranged to receive power, e.g., from the AC mains, for wirelessly charging a battery of the portable electronic device 100, 200 when the portable electronic device 100, 200 is coupled to the station. The station 400 preferably includes an AC power cable and plug for connecting to the AC mains socket.

During operation, the base station 400 is operable to charge, preferably wirelessly charge, the portable electronic device 100, 200 when the device 100, 200 is electrically coupled to or engaged with station. Regardless of whether the portable electronic device 100, 200 is attached to the base station 400, the base station 400 is operable to receive data, in real time or at regular time intervals, from the portable electronic device 100, 200 through the wireless communication modules 206, 406 on the two devices. The base station 400 may store the data received, and may, in real time or at regular time intervals, transmit the data received to a remote server for further storage or analysis.

In some embodiments, the base station 400 may receive commands or data from the remote server. The base station 400 may relay these commands or data to the portable electronic device 100, 200, preferably wirelessly, to alter the configuration or data in the device. In one example, the alarm command may be sent from the remote server to the portable electronic device 100, 200 through the base station 400.

In some embodiments, the base station 400 may further include one or more sensors operably connected with the processor 402, memory 404, communication module 406 and the power module 408. The sensors may include an air quality sensor, a temperature sensor, a humidity sensor, etc. The air quality sensor may be arranged to detect quality of air in the environment in which the base station 400 is arranged. In one example, the air quality sensor is a CO₂ sensor or a VOC sensor. The temperature sensor and the humidity sensor may be used to detect temperature and humidity in the environment in which the base station 400 is arranged. Information obtained from the sensors could be processed by the processor 402, stored in the memory 404, or transmitted to other external electronic devices through the communication module 406.

In some embodiments, the base station 400 may further include one or more additional functional modules operably connected with the processor 402, memory 404, communication module 406 and the power module 408. The additional functional modules may be a nightlight, for example multicolored nightlight. The additional function module may be Bluetooth or Wi-Fi gateway, allowing the base station 400 to be used as a router or wireless communication control hub.

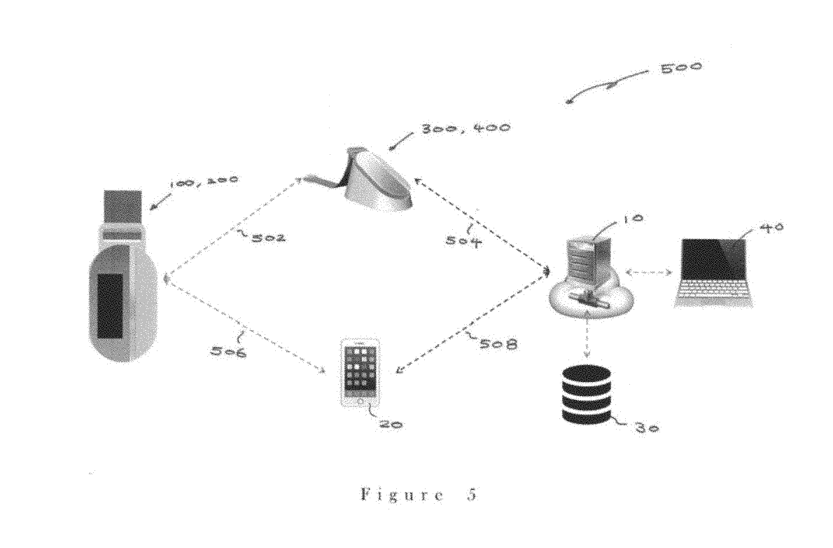

FIG. 5 shows a system architecture 500 in which the portable electronic device 100, 200 and the charging station 300, 400 in accordance with some embodiments of the present invention may operate in.

As shown in FIG. 5, the portable electronic device 100, 200 can be operably connected (e.g., in data communication) with a base station 300, 400 through a first wireless communication link 502, and the base station 300, 400 can in turn be operably connected (e.g., in data communication) with a remote server 10 through a second data communication link 504. The first communication link 502 is preferably a BLE communication link, and the second communication link 504 is preferably a Wi-Fi communication link. The first and second communication links 502, 504 are preferably bi-directional, and encrypted.

The portable electronic device 100, 200 can also be operably connected (e.g., in data communication) with a mobile phone 20 (with a corresponding application installed) through a third wireless communication link 506, and the mobile phone 20 can in turn be operably connected (e.g., in data communication) with the remote server 10 through a fourth data communication link 508. The third communication link 506 is preferably a BLE communication link, and the fourth communication link 508 is preferably a Wi-Fi or telecommunication (e.g., 2G, 3G, 4G, LTE) communication link. The third and fourth communication links 506, 508 are preferably bi-directional, and encrypted.

The remote server 10 may be further connected or associated with a database 30 for storing data. A computer 40, in the form of a notebook in this embodiment, is connected with the server 10 for obtaining data from or sending data to the server 10. The server 10 may be a cloud server.

In operation, the data obtained by the portable electronic device 100, 200 may be transmitted in real time or at regular time intervals, to either the base station 300, 400 or the mobile phone 20. Preferably, the mobile phone 20 is installed with an application (e.g., interface) that allows the user to readily interact with the portable electronic device 100, 200. The base station 300, 400 or the mobile phone 20 may store the information received form the portable electronic device 100, 200, and they may relay the data to the application server 10. Data at the server 10 can be stored in a database 30, and may be accessed by a remote computing device 40, e.g., notebook. Data analysis can preferably be performed on the mobile phone 20, in the server 10, or on the notebook 40. On the other hand, commands or data (e.g., alarm setting command) may be transmitted to the portable electronic device 100, 200 from the smart phone 20, or from the notebook 40 through the server 10 and either the mobile phone 20 or the base station 300, 400.

In FIG. 5, the server 10, mobile phone 20, and notebook 40 can generally be referred to as “information handling systems”. In general, these information handling systems may each comprise suitable components necessary to receive, store and execute appropriate computer instructions or codes. The main components of the system are a processing unit and a memory unit. The processing unit is a processor such as a CPU, an MCU, or a microprocessor. The memory unit may include a volatile memory unit (such as RAM), a non-volatile unit (such as ROM, EPROM, EEPROM and flash memory) or both. Preferably, the information handling system further includes one or more input devices such as a keyboard, a mouse, a stylus, a microphone, a tactile input device (e.g., touch sensitive screen) and a video input device (e.g., camera). The system may further include one or more output devices such as one or more displays, speakers, disk drives, and printers. The displays may be a liquid crystal display, a light emitting display or any other suitable display that may or may not be touch sensitive. The system may further include one or more disk drives 212 which may encompass solid state drives, hard disk drives, optical drives and/or magnetic tape drives. A suitable operating system may be installed in the system, e.g., on the disk drive or in the memory unit of the system. The memory unit and the disk drive may be operated by the processing unit. The system also preferably includes a communication module for establishing one or more communication links (not shown) with one or more other computing devices such as a server, personal computers, terminals, wireless or handheld computing devices. The communication module may be a modem, a Network Interface Card (NIC), an integrated network interface, a radio frequency transceiver, an optical port, an infrared port, a USB connection, or other interfaces. The communication links may be wired or wireless for communicating commands, instructions, information and/or data. Preferably, the processing unit, the memory unit, and optionally the input devices, the output devices, the communication module, and the disk drives are connected with each other through a bus, a Peripheral Component Interconnect (PCI) such as PCI Express, a Universal Serial Bus (USB), and/or an optical bus structure. In one embodiment, some of these components may be connected through a network such as the Internet or a cloud computing network. A person skilled in the art would appreciate that the system shown in FIG. 5 is merely exemplary and that different information handling systems may have different configurations and still be applicable in the present invention.

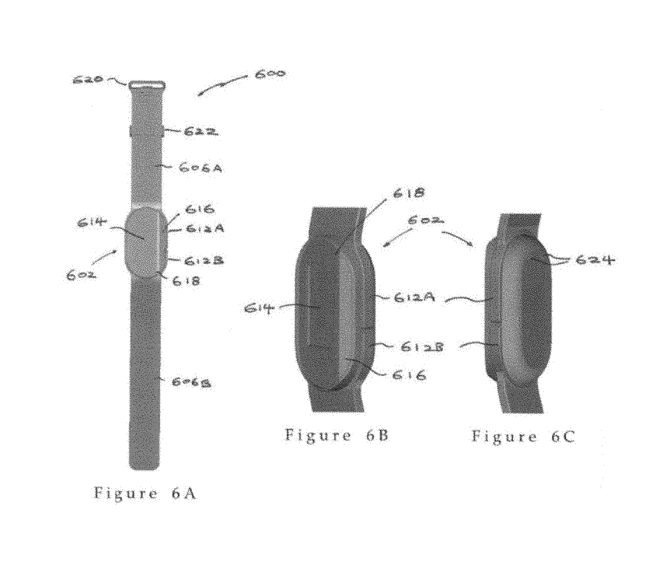

FIGS. 6A-6C show an exemplary portable electronic device 600 in accordance with another embodiment of the present invention. The device 600 includes a housing 602 having a substantially oblong profile. Two strap sections 606A, 606B are mounted at opposite ends of the housing 602, for example, through hinges at respective strap attaching portions of the housing 602. Preferably, the device 600 has the form of a watch. In one embodiment, the first strap section 606A may include a buckle 620 attached at one end and a free loop 622 freely slidable along the first strap section 606A. The second strap section 606B may include adjustment holes (not shown) disposed along a length of the strap section 606B for receiving the buckle tongue of the buckle 620 when the device 600 is worn by the user. The two strap sections 606A, 606B may be made of plastic, leather, or metal. The strap sections 606A, 606B and the housing 602 together define a closed loop, the size of which is adjustable, allowing the device 600 to be worn by different users with different wrist sizes.

As shown in FIGS. 6B and 6C, two buttons 612A, 612B are arranged on the same long edge of the housing 102. In this embodiment, the first button 612A covers approximately two-thirds of the length of the edge and the second button 612B covers approximately one-third of the length of the edge. Preferably, the buttons 612A, 612B are momentary actuators arranged to be pressed by the user to receive a user input. Unlike the buttons 112A-112D in the embodiment of FIG. 1, the buttons 612A, 612B in the present embodiment are not associated with specific types of task-related event related to care needs of a subject. Rather, in operation of the present embodiment, the first button 612A is dedicated for menu activation, new record creation, and confirmation for sending data; and the second button 612B is dedicated to cancel function or return-to-main-menu function.

As shown in FIGS. 6A and 6B, the front side of the housing 600 includes a display 614, a touch panel 616, and a light indicator bar 618 separating the display 614 and the touch panel 616. The display 614 is preferably a LCD display screen. Optionally, the display 614 may be touch-sensitive for receiving user input. In operation, the display 614 may display data, selections, instructions, etc., in the form of text or graphics, to assist data entry. The touch panel 616 is operable, during operation, to receive user input for event categories, event attributes, and event values, in the form of tapping, pinching, sliding, or swiping, etc. as performed by the user on the panel 616. The light indicator bar 618 in the present embodiment is an LED array that includes LEDs of different colours, e.g., red, green, and blue. The light indicator bar 618 is arranged to provide a visual alarm or to display different status of the device (e.g., low power state, error state, etc.).

In the present embodiment, the user can input data relating to time-related or task-related events associated with care needs of a subject, biological events related to the subject, and biological measurements related to the subject, using the buttons 612A, 612B and the touch panel 616 (based on instructions shown on the display) of the device 600. For an infant subject, the time-related or task-related event may be a diaper change event, a feeding event, a sleeping event, or a medication event. The diaper change event may include a time at which a diaper change occurs, a stool event, or an urination event. The stool or urination event may include stool colour, stool quantity, stool quality, urine colour, urine quantity, or urine quality. The feeding event may include a time at which feeding begins or a time at which feeding ends, a feeding volume, or a feeding source (e.g., formula, breastmilk, breastfeed-left, breastfeed-right, solids). The sleeping event may include a time at which sleeping begins or a time at which sleeping ends. The medication event may include the time at which medication is taken, medication volume, or medication source. In one embodiment, the device 600 is also operable record a breastmilk expression event (pumping) which includes, for example, the time at which the expression occurs or the volume of the breastmilk expressed.

As shown in FIG. 6C, two conductive charging pins 624, operably connected with a power source arranged in the housing 602, are arranged at the back side of the housing 602. In the present embodiment, the charging pins 624 are spring biased pogo pins that, when actuated (e.g., contacted and depressed against the spring bias force), allow power communication from an external power source (e.g., charger) to the power source inside the housing 602. Preferably, respective seals are provided around the pins 624 to prevent dirt or water from entering the housing 602. It should be noted that in other embodiments any number of charging pins may be used and the charging pins need not be pogo pins.

The portable electronic device 600 of FIGS. 6A-6C further includes a number of internal components (not visible from FIGS. 6A-6C). In the present embodiment, the device 600 includes a microprocessor, a memory, and a wireless communication module that are operably connected with each other through a main bus.

The microprocessor is arranged, during operation, to process program instructions for display on the display 614 to interact with the user and to process user inputs received through the buttons 612A, 612B and the touch panel 616.

Preferably, the memory of the device 600 comprises a non-volatile memory for storing data inputted by the user. Data stored in the non-volatile memory is substantially unaffected by the power state of the device 600. In one example, when the user presses the button 612A, 612B or manipulates the touch panel 616 to record an event or measurement entry (time-related or task-related events associated with care needs of a subject, biological events related to the subject, or biological measurements related to the subject), the corresponding data or entry is stored in the memory. The memory in the present embodiment is arranged to store historical data, as long as the memory capacity has not been exceeded. In one example, the memory capacity is sufficient for storing event records of a few days or one week. Data stored in the memory may be transferred to an external electronic device and hence to a server through the wireless communication module of the device 600. In some embodiments, data that have been transferred to the server can be overwritten in subsequent recording operations. The memory in the present embodiment may also comprise other volatile or non-volatile memory for storing program instructions or parameters.

Like the embodiment of the device 100 in FIGS. 1A-2, the wireless communication module in the device 600 of the present embodiment is also a Bluetooth Low Energy (BLE) communication module arranged to communicate with the external electronic device using Bluetooth Low Energy communication protocol. The BLE communication module is arranged to communicate data in real time to and from the device 600, but in some cases, it may communicate data at regular time intervals. In one example, the BLE communication module is arranged to communicate in real time the data or the new data stored in the memory to an external electronic device and hence to a server. The BLE communication module may also receive commands, program instructions, or data from an external electronic device to affect the function of the device. Preferably, the communication is secured and encrypted.

The portable electronic device 600 of FIGS. 6A-6C further includes, internally, a power source. In the present embodiment, the power source includes a battery that is rechargeable and may have Lithium-based chemistry. Optionally, the battery is hard-wired inside the housing, i.e., it is neither removable nor replaceable by the user. The power source also includes a charging circuit in electric connection with the battery and the charging pins 624. The charging pins 624 may receive power from an external charging station, through direct contact with corresponding charging pins or plugs on the charging station. The power source in the present embodiment is operably connected with different functional modules of the device to provide power to the different functional modules.

In the present embodiment, an alarm module is further provided inside the portable electronic device 600. The alarm module is arranged to be controlled by the processor to provide alarm function—to remind the user of the need to perform a specific task at a specific moment. The alarm module in the present embodiment is similar to that in the device 100, 200 of FIG. 1A-2—it includes one or more of a visual alarm module, a tactile alarm module, and an audible alarm module, that may operate individually or together to provide different alarms. Details of the alarm module will not be repeated here. In the present embodiment, the alarm may only be set by the user locally at the device 600 using the buttons 612A, 612B and/or the panel 616, i.e., the 600 device is not arranged to receive time-related reminder data from other external electronic devices through the communication module. In one embodiment, the alarm-related data associated with the alarm that is set at the device 600 may be communicated through the communication module to external electronic device and hence to a phone application or website. Restricting the alarms to be set only locally at the device can ensure that the device 600 is the master source of alarm-related data, avoiding confusion and minimizing the chance of the alarms being re-configured without the user's knowledge.

In some embodiments, the portable electronic device 600 may include further functional modules operably connected with the other modules in the device 200. For example, the device 200 may include a GPS module for tracking geographical location of the user wearing the device 200.

In the present embodiment, to record an event during operation, the user first presses the first button 612A to activate (e.g., “wake-up”) the device 600. Upon activation, a menu according to the program instructions stored will be shown on the display 614. Preferably, the menu is arranged to display event categories to be selected by the user. The event categories may include any of the following: “diaper change event”, “feeding event”, “sleeping event”, “medication event”, and “breastmilk expression event”. The user may, through swiping and tapping on the touch panel 616, select the event category to be recorded. In one example, a first swipe will bring up the “diaper change event” category, a second swipe will bring up the “feeding event” category, etc. When the desired category is shown, the user may tap on the panel 616 to make a selection. In some embodiments, there may be subcategories under a category. For example, the “diaper change event” category may include the subcategories of “the time at which a diaper change occurs”, “stool event”, or “urination event”. Further sub-categories are possible in some embodiments. In any case, the user has to manipulate the touch panel 616 to make a selection. Preferably, once a selection is made, one or more previous recorded entries associated with the respective selected event category or subcategory are displayed on the display 614 so that the user can readily view of the most recent entries. After selecting the event category or subcategory to be recorded, the user presses the first button 612A to create a new entry. The user may, through manipulating the touch panel 616, enter values (time, number, etc.) associated with the event or measurement to the device 600. Once the correct value has been entered, the user then again actuates the first button 612A to record the event or measurement. At any time during operation, to cancel an entry or to return to the main menu, the user may simply press the second button 612B. In operation of the present embodiment, the user has to press the button 612A three times to make an entry.

Other aspects of the device 600 in the present embodiment are similar to the devices 100, 200 described with respect to FIGS. 1A-2, and so are not further discussed.

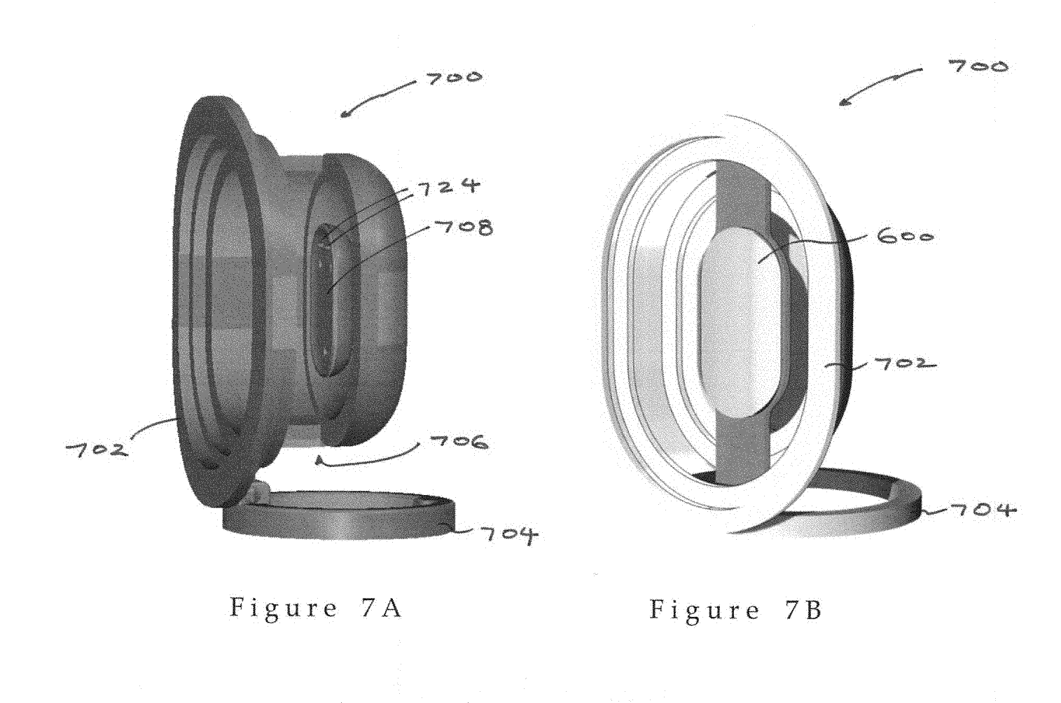

FIGS. 7A and 7B show an exemplary base station 700 suitable for charging the portable electronic device 600 of the present invention. In FIG. 7A, only the base station 700 is shown; in FIG. 7B, the portable electronic device 600 is shown mounted to the base station 700.

As shown in FIGS. 7A and 7B, the base station 700 in the present embodiment includes a body 702 hingedly connected to a base 704. The body 702 defining a space with a lateral opening 706 through which the device 600 may be inserted from one side of the body. A docking portion 708 is arranged in the body 702 for mounting the portable electronic device 600. The docking portion 708 preferably includes a magnetic mount, for example, with a magnet (not shown) for attracting a corresponding metallic or magnetic part or member arranged in the portable electronic device 600. The docking portion 708 also includes charging pins or contacts 724 in connection with a charging circuit inside the body 702. The charging pins or contacts 724 of the station 700 are sized and arranged to correspond to the charging pins or contacts 624 on the device 600 for establishing electrical connection between the base station 700 and the device 600 for charging the device 600. The station 700 also includes an AC power cable and plug (now shown) for connecting to the AC mains socket to receive power. FIG. 7B shows the device 600 mounted to the charging station 700.

In the present embodiment, the base station 700 includes, internally, a processor for processing data, a memory for storing data, and one or more wireless communication modules arranged to communicate data. Like the stations 300, 400 described with respect to FIGS. 3 and 4, the wireless communication module(s) of the station 700 in the present embodiment may communicate with the wireless communication module of the portable electronic device 600 and may further communicate data with a remote server.

Preferably, the base station 700 may further include one or more other sensors or detectors for extended functionality. For example, the base station 700 may include a temperature sensor for detecting ambient temperature. In one embodiment, the detected temperature may be recorded in the memory and optionally transmitted to a remote server through the wireless communication module. The base station 700 may include one or more air quality sensors that can detect the level of benzene (e.g., from tobacco cigarette), CO₂ (indicates ventilation of the room), CO, or other gases in air. In one embodiment, the detected air quality measure may be recorded in the memory and optionally transmitted to a remote server remote server through the wireless communication module. Other sensors for detecting humidity, odor, ambient sound, ambient light, etc., can also be incorporated into the station. In one example, the station 700 may include one or more displays or light indicators for displaying the detected result or warning (if the detected level exceeds a predetermined threshold). In one embodiment, the station 700 may include a light, e.g., in the form of LEDs, to provide night-light feature to aid the subject's (e.g., infant) sleeping habits.

Other aspects of the base station 700 in the present embodiment are similar to those described with respect to the stations 300, 400 of FIGS. 3 and 4 (for example, the additional sensors and functional modules in some embodiments), and so are not further discussed.

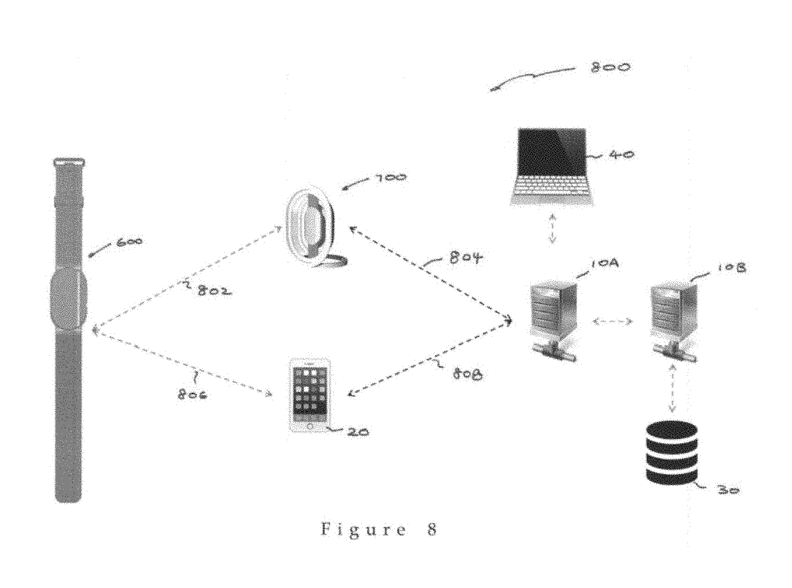

FIG. 8 shows a system architecture 800 in which the portable electronic device 600 and the charging station 700 in accordance with some embodiments of the present invention may operate in.

As shown in FIG. 8, the portable electronic device 600 can be operably connected (e.g., in data communication) with a base station 700 through a first wireless communication link 802, and the base station 700 can in turn be operably connected (e.g., in data communication) with a remote server 10A, 10B through a second data communication link 804. The first communication link 802 is preferably a BLE communication link, and the second communication link 804 is preferably a Wi-Fi communication link. The first and second communication links 802, 804 are preferably bi-directional, and encrypted.

The portable electronic device 600 can also be operably connected (e.g., in data communication) with the mobile phone 20 (with a corresponding application installed) through a third wireless communication link 806, and the mobile phone 20 can in turn be operably connected (e.g., in data communication) with the remote server 10A, 10B through a fourth data communication link 808. The third communication link 806 is preferably a BLE communication link, and the fourth communication link 808 is preferably a Wi-Fi or telecommunication (e.g., 2G, 3G, 4G, LTE) communication link. The third and fourth communication links 806, 808 are preferably bi-directional, and encrypted.

The remote server 10A, 10B may include a web server 10A and an application server 10B in data communication with one another. Although in the present embodiment the web server 10A and application server 10B are shown as two separate servers, it should be appreciated that in other embodiments the web server 10A and application server 10B may be implemented using various hardware and software, and so their number need not be two. In the present embodiment, a computer 40, in the form of a notebook, is connected with the web server 10A for obtaining data from or sending data to the web server 10A. The web server 10A and application server 10B may be a cloud server distributed on a cloud computing network. The application server 10B is connected or associated with a database 30 for storing data.

In operation, the data obtained by the portable electronic device 600 may be transmitted in real time or at regular time intervals, to either the base station 700 or the mobile phone 20. Preferably, the mobile phone 20 is installed with an application (e.g., interface) that allows the user to readily interact with the portable electronic device 600. The base station 700 or the mobile phone 20 may store the information received form the portable electronic device 600, and they may relay the data to the servers 10A, 10B. Data received at the server can be stored in a database 30, and may be accessed by a remote computing device 40, e.g., notebook. Data analysis can preferably be performed on the mobile phone 20, on the servers 10A, 10B, or on the notebook 40. On the other hand, commands or data may be transmitted to the portable electronic device 600 from the smart phone 20, or from the notebook 40 through the server 10A, 10B, and either the mobile phone 20 or the base station 700.

In FIG. 8, the server 10A, 10B, mobile phone 20, and notebook 40 are similar to the “information handling systems” described with respect to FIG. 5.

Preferred embodiments of the portable electronic device, the charger, and the system of the present embodiment have been described above with reference to the drawings.

A person skilled in the art would appreciate that the portable electronic device of the present invention is not limited to the form too, 200, 600 as illustrated in FIGS. 1A, 1B, 2, and 6A-6C. Instead, the portable electronic device of the present invention may be any portable, handheld, or wearable electronic device that has a housing with a display for displaying instructions or data; and a plurality of actuators and a touch panel, for receiving user input associated with one or more of: time-related or task-related events associated with care needs of a subject, biological events related to the subject, and biological measurements related to the subject. The housing of the device contains a processor operably connected with the plurality of actuators and the touch panel; a memory operably connected with the processor; and a wireless communication module operably connected with the memory and/or the processor. During operation, the processor is operable to process the user input received at the plurality of actuators and the touch panel for recording the respective event or measurement; and the memory is arranged to store the recorded event or measurement as data for transmission through the wireless communication module to an external electronic device. The device of the present invention is a dedicated device. It may have different configurations, and it generally comprises suitable components necessary to receive, store and execute appropriate computer instructions or codes. Different features of the various embodiments of the portable electronic device may be combined, re-arranged, or modified, to form new embodiments which are still within the scope of the present invention.

Likewise, a person skilled in the art would appreciate that the charging station for the portable electronic device of the present invention is not limited to the form 300, 40, 700 as illustrated in FIGS. 3, 4, and 7A-7B. Instead, the charging station of the present invention may be any charging station, suitable for charging and communicating data with the portable electronic device, having a body arranged to provide a mount for the portable electronic device. The body contains a processor for processing data, and a wireless communication module arranged to communicate data with the wireless communication module of the portable electronic device and to communicate data with a server. The station may include additional structural or functional modules, e.g., input/output devices, such as display, buttons, speakers, etc., for receiving user input and displaying information and data to the user, and sensors and detectors for extended functionality. Different features of the various embodiments of the charger may be combined, re-arranged, or modified, to form new embodiments which are still within the scope of the present invention.

The system in which the portable electronic devices and base stations of the present invention can operate is not limited to the form 500, 800 illustrated in FIGS. 5 and 8. Instead, the system may include the portable electronic device in accordance with some embodiments of the present invention, and an external electronic device operably connected with the portable electronic device. Different features of the various embodiments of the system may be combined, re-arranged, or modified, to form new embodiments which are still within the scope of the present invention.

In one embodiment, the system 500, 800 may further include other standalone electronic devices (not shown) such as a smart speaker, a thermometer, a scale, an IP camera, etc., all of which can be operably connected to the base station 300, 400, 700 and/or the device 100, 200, 600. The smart speaker may be arranged to provide voice-controlled data entry. The speaker may also be used to report baby activities, play music, white noise, etc. The speaker can be used for monitoring baby activities too (e.g., detect when the baby is crying). The thermometer may be wearable or handheld. The scale may be used to performing height and weight measurement, mainly for infants and babies. The IP camera is to provide video and audio monitoring. In some applications, the IP camera may include movement sensor for detecting movement of the infants or babies. Data from all these devices can be sent to the server (e.g., cloud server) for storage and analysis. All information may also be made readily available to the user through the user interface of the electronic device 100, 200, 600 and/or the mobile phone 20 (or like electronic devices such as tablet, computer, laptop).

One of the key aspects of this invention is to combine real-life caretaking experience with technology to develop a practical system that eases the effort for caretakers recording the development events. For infant subjects, parents and caretakers have to frequently communicate regarding when did the child last sleep, when was the last diaper change, how much and when did the child last feed as well as other enquires to track the child's health, and there is a large amount of information and data points that need to be effectively communicated and stored to ensure and promote the wellbeing of the infant.

Some embodiments of the present invention provide a dedicated portable electronic device that is lightweight, uses BLE communication, designed to be worn or held by the caretaker, to enhance ease and timeliness of use.

The handheld electronic device of the present invention can be worn or readily carried by the caretaker who may use it for:
setting reminders, which may then be sent through an application on a mobile phone or through a website to a server, to perform predetermined tasks or checks;
upon checking and/or completing the task, such as changing a diaper, the caretaker can record the time and date of the event by simply pushing a button on the device. If needed, other details related to the specific event, such as the colour of bowel movement, can be input using a variable control. Use of the physical buttons and LCD screen on the wireless device will allow the caretaker to create and select the relevant event for recording as well as retrieve information on previous events without using other electronic devices, thereby minimizing distractions.

In some embodiments of the present invention, once the data is recorded on the portable electronic device, the data points are stored in the non-volatile memory before wirelessly transmitting the data to a secure application server on the internet. As the portable electronic device may only have a BLE radio, which helps to save power and minimize Specific Absorption Rates (“SAR”), it may have to be connected to the web or application server using one or both of the following:
Mobile phone (or tablet) application: the application will act as the gateway between the wearable connecting to the mobile phone via BLE and use the mobile phone network (2G, 3G, 4G, LTE, 5G, etc.) or Wi-Fi to connect to the application Server;
Base Station: the base station can provide an alternate gateway to the application server on the internet, and may further function as a wireless charging station for the portable electronic device.

Once the data reaches the application server, it is then collated and stored in a database for analysis and presentation on demand by suitable persons (e.g., parents, doctors, etc.) any time, any place on any device that can access a website associated with the server. Both the website and the mobile phone application can provide rich visualizations of the data points, showing current and past events, and create automated comparisons, e.g., against growth and development rates set by the World Health Organization. In some embodiments, the data points may be used to produce predictive trends associated with individual subjects.

It should not note that in some embodiments the subject that requires care is an infant. However, in some other embodiments of the present invention, the subject may be an elderly, a patient, or any other subject that require care. The subject is preferably a human but may also be other animals.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A portable electronic device comprising:
a housing with
a display for displaying instructions or data; and
a plurality of actuators and a touch panel, for receiving user input associated with one or more of: time-related or task-related events associated with care needs of a subject, biological events related to the subject, and biological measurements related to the subject;
the housing contains
a processor operably connected with the plurality of actuators and the touch panel;
a memory operably connected with the processor; and
a wireless communication module operably connected with the memory and/or the processor;
wherein during operation the processor is operable to process the user input received at the plurality of actuators and the touch panel for recording the respective event or measurement; and
wherein the memory is arranged to store the recorded event or measurement as data for transmission through the wireless communication module to an external electronic device.

2. The portable electronic device in accordance with claim 1, wherein the task-related event, time-related event, biological event, and biological measurement comprises any of:
a diaper change event;
a feeding event;
a sleeping event; and
a medication event.

3. The portable electronic device in accordance with claim 2, wherein the diaper change event comprises any of:
a time at which a diaper change occurs, and
a stool or urination event.

4. The portable electronic device in accordance with claim 2, wherein the stool or urination event comprises any of:
stool colour,
stool quantity,
stool quality,
urine colour,
urine quantity, and
urine quality.

5. The portable electronic device in accordance with claim 2, wherein the feeding event comprises any of:
a time at which feeding begins,
a time at which feeding ends,
a feeding volume, and
a feeding source.

6. The portable electronic device in accordance with claim 2, wherein the sleeping event comprises:
a time at which sleeping begins or a time at which sleeping ends

7. The portable electronic device in accordance with claim 2, wherein the medication event comprises any of:
a time at which medication is taken,
a medication volume, and
a medication source.

8. The portable electronic device in accordance with claim 1, wherein the user input is further associated with a breastmilk expression event comprising any of:
a time at which the expression occurs, and
a volume of the breastmilk expressed.

9. The portable electronic device in accordance with claim 1, wherein the plurality of actuators are operable as selection keys during operation.

10. The portable electronic device in accordance with claim 1, wherein the housing further comprising an alarm module operably coupled with the processor for providing an alarm.

11. The portable electronic device in accordance with claim 10, wherein the alarm module comprises one or more of: a visual alarm module, a tactile alarm module, and an audible alarm module.

12. (canceled)

13. The portable electronic device in accordance with claim 11, wherein the tactile alarm module comprises a vibration motor.

14. The portable electronic device in accordance with claim 11, wherein the audible alarm module comprises a speaker or a buzzer.

15. The portable electronic device in accordance with claim 1, wherein the wireless communication module comprises a Bluetooth Low Energy communication module arranged to communicate with the external electronic device using Bluetooth Low Energy communication protocol.

16. The portable electronic device in accordance with claim 1, wherein the plurality of actuators are momentary actuators.

17. (canceled)

18. (canceled)

19. The portable electronic device in accordance with claim 1, the housing further contains a power source.

20. (canceled)

21. (canceled)

22. (canceled)

23. (canceled)

24. The portable electronic device in accordance with claim 1, wherein the housing has a substantially oblong profile, and the plurality of actuators are arranged along a perimeter of the housing.

25. The portable electronic device in accordance with claim 1, wherein the subject is an infant.

26. A charging station for the portable electronic device of claim 1, comprising:
a body arranged to provide a mount for the portable electronic device;
the body containing:
a processor for processing data; and
a wireless communication module arranged to communicate data with the wireless communication module of the portable electronic device and to communicate data with a server.

27. The charging station in accordance with claim 26, wherein the body further contains a charging circuit, arranged to receive power, for charging a power source of the portable electronic device.

28. The charging station in accordance with claim 26, wherein the body further contains a memory for storing data received from the portable electronic device and/or data received from the server.

29. The charging station in accordance with claim 26, wherein the wireless communication module comprises a Bluetooth Low Energy communication module arranged to communicate data with the portable electronic device using Bluetooth Low Energy communication protocol.

30. The charging station in accordance with claim 26, wherein the wireless communication module comprises a Wi-Fi communication module arranged to communicate data with the server using Wi-Fi communication protocol.

31. A system comprising:
a portable electronic device in accordance with claim 1; and
an external electronic device operably connected with the portable electronic device.

32. The system in accordance with claim 31, wherein the external electronic device is a charging station or a mobile device comprising:
a body arranged to provide a mount for the portable electronic device;
the body containing:
a processor for processing data; and
a wireless communication module arranged to communicate data with the wireless communication module of the portable electronic device and to communicate data with a server.

33. The system in accordance with claim 32, further comprising:
a server operably connected with the charging station, wherein the server is operable to store, process, or analyze data received from the charging station.

34. The system in accordance with claim 33, further comprising at least one of the following components operably connected with the server: a speaker, a thermometer, a scale, and a camera.
